(19) United States
(12) Patent Application Publication
(10) Pub. No.: US 2008/0114550 A1
(43) Pub. Date: May 15, 2008

(54) Cell density fitting equation

(76) Inventors: Barbara Paldus, Woodside, CA (US); Mark Selker, Los Altos, CA (US)

Correspondence Address: Herbert Burkard, BLDG.1, 3350 Scott Blvd., Santa Clara, CA, 95054, US

(21) Appl. No.: 11/702,861

(22) Filed: Feb. 6, 2007

Related U.S. Application Data
(60) Provisional application No. 60/858,329, filed on Nov. 10, 2006.

(51) Int. Cl. G01N 33/48

(52) U.S. Cl. 702/19

(57) ABSTRACT

A method for converting non-linear optical loss readings in a bioreactor process vessel into process parameter units by applying a curve fitting algorithm to the fitting function represented by the empirical equation:

$$yol = A + B ( 1 - {e}^{- \frac{{x}_{PU}}{C}} ) + D \cdot {x}_{PU}$$

wherein x_{PU} is in the process units (PU), yₒₗ is optical loss in the chosen units, A is the offset, D is the absorption coefficient, B is the effective scattering coefficient, and C is the scattering constant. The preferred fitting algorithm is Levenberg-Marquardt.

### RELATED APPLICATIONS

This application claims priority from pending provisional application Ser. No. 60/858,329, filed Nov. 10, 2006. The invention disclosed and claimed herein is related to the sensor designs described in co-pending applications Ser. Nos. 60/835329, 10/856,885, 11/139,720 and 11/002,021 the disclosures of which are incorporated herein by this reference.

### FIELD OF THE INVENTION

This invention is directed to a method for improving the accuracy of the optical density (absorption) sensor measurements such as are utilized in connection with the control of process parameters in bioreactors.

### BRIEF DESCRIPTION OF THE DRAWINGS

FIG. 1a and 1b illustrate optical absorption principles: FIG. 1a illustrates reference and sample measurement and FIG. 1b illustrates samples having increasing absorbance values.

FIG. 2 shows the absorption coefficient of pure water as a function of incident light wavelength.

FIG. 3 shows a calibration curve of concentration versus absorbance for aqueous copper sulfate solutions.

FIG. 4 shows the typical sizes of biological particles and the scattering mechanism they would produce with a sensor using incident light having a wavelength of 830 nm.

FIGS. 5(a) and (b) show scattering loss measured as a function of particle concentration with light at 830 nm over a 10 mm path length. The media consists of 3 micron polystyrene spheres dispersed in water, FIG. 5(a) shows optical losses lower than 1 AU, and FIG. 5(b) optical losses exceeding 1 AU.

FIG. 6 shows a Curve Fit Program Screen.

FIG. 7 shows original fit versus fit with worse case 0.1% error in all parameters.

FIG. 8 shows the response of Optical Density in AU versus Yeast % Solids.

FIG. 9 shows the response of Optical Density in AU versus E. Coli Optical Density.

FIG. 10 shows the response of Optical Density in AU versus Chinese hamster Ovary (CHO) cell concentration.

FIG. 11 shows the response of Optical Density in AU versus Formazin Concentration.

### BACKGROUND OF THE INVENTION

### Optical Loss

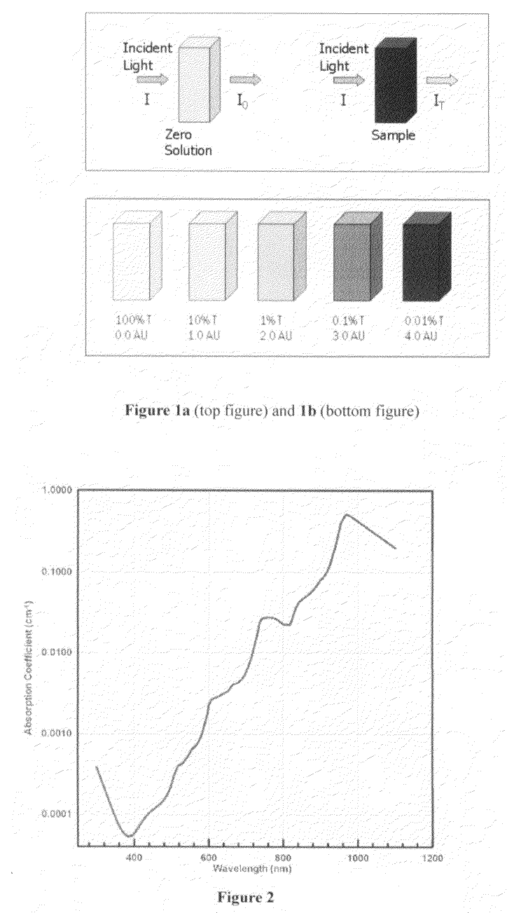

An optical density sensor measures the reduction in the transmission of the light (called “optical loss”) as it passes across the measurement gap of an optical density probe. The measurement gap defines the optical path length (OPL) across which the light passes when the probe tip is placed in a liquid medium such as a fermentation broth. As the optical loss increases, the amount of light transmitted across the optical gap decreases as shown in FIGS. 1a and 1b. The standard measurement unit of optical loss, Lₒₚₜ, is the absorbance unit (AU).

Lₒₚₜ depends on the wavelength, λ, of the light, and is given by:

$$\begin{matrix}{L}_{opt} ( λ ) = A ( λ ) + S ( λ ) + {L}_{other} ( λ ) \\ = - {log}_{10} ( \frac{{I}_{T} ( λ )}{{I}_{0} ( λ )} ) [ AU ]\end{matrix} \qquad Equation ( 1 )$$

In which: I_{T}(λ)=Light transmitted through a sample at wavelength λ
I₀(λ)=Light transmitted through a zero/reference solution at wavelength λ
A(λ)=Optical loss from absorption, also called absorbance, at wavelength λ
S(λ)=Optical loss from scattering at wavelength λ, and
Lₒₜₕₑᵣ(λ)=Optical loss from non-linear effects or measurement processes at wavelength λ.

### Absorbance

Absorbance, A(λ), is a measure of the conversion of radiant energy to heat and chemical energy. It is numerically equal to the fraction of energy absorbed from a light beam over an OPL traveled in an absorbing medium. The Beer-Lambert law defines a linear relationship between A(λ) and the OPL, through the molar extinction coefficient, μ_{α}(λ, Δ), and absorption coefficient, α(λ, Δ):

A(λ)=μ₆₀ (λ, Δ)*OPL=α(λ, Δ)/ln(10)*OPL Eq. (2)

The molar extinction and absorption coefficients have units of cm⁻¹, and are proportional to the concentration of the absorbing species, Δ, through the extinction coefficient, ε(λ) namely, α(λ, Δ)=ε(λ)*Δ. Therefore, for a fixed concentration of absorbing species, absorbance and OPL are proportional, and the slope of the line is the sample's molar extinction coefficient, μ_{α}. The absorption coefficient can also be related to the number density of absorber molecules, N, through the absorption cross-section, σ_{abs}, namely:

α(λ, N)=σ_{abs}(λ)*N Eq. (3)

FIG. 2 shows that the absorption coefficient of pure, particle-free water ranges from 0.03-0.06 cm⁻¹ when the incident light is in the 760-1000 nm wavelength band. At 830 nm, (a wavelength of minimum absorption for water and hence a preferred operating wavelength for an optical density sensor for aqueous media measurements) the absorption coefficient is 0.03 cm⁻¹, which means that for a 1 cm OPL, A=0.03 AU. Because distilled, deionized water is used as the zero reference liquid, the absorbance of water is virtually negated in such optical density measurements.

In the case of a purely absorbing sample, absorbance is obtained by measuring the attenuation of the light as it passes through the sample. Namely, absorbance is governed by the following equation:

Aλ=Absorbance=−log₁₀(I_{T}/I₀)[AU]=−log₁₀(T)[AU] Eq. (4)

In which: I_{T}=Light transmitted through a sample, and
I₀=Light transmitted through a zero/reference solution
T=Light transmittance expressed as decimal percent

Absorbance is calculated from the measurement of light transmitted through the sample and referenced to a zero solution. For increasing concentrations of the absorbing medium, the amount of the transmitted light decreases, and the absorbance value increases. Modern spectrometers usually display measured data as either transmittance, %-transmittance, or absorbance. Spectrometer cuvettes used for absorption measurements often have an OPL of 1 cm, so that the absorption coefficient and absorbance readings can be directly compared.

An unknown concentration, Δ, of a purely absorbing analyte can be determined by measuring the amount of light that a sample absorbs and applying the Beer-Lambert law:

Δ[M]=A[AU]/ε[M¹ cm⁻¹]/OPL[cm] Eq. (5)

If the absorption coefficient for a given analyte is not known, the unknown concentration can be determined using a working curve of absorbance versus concentration derived from reference standards.

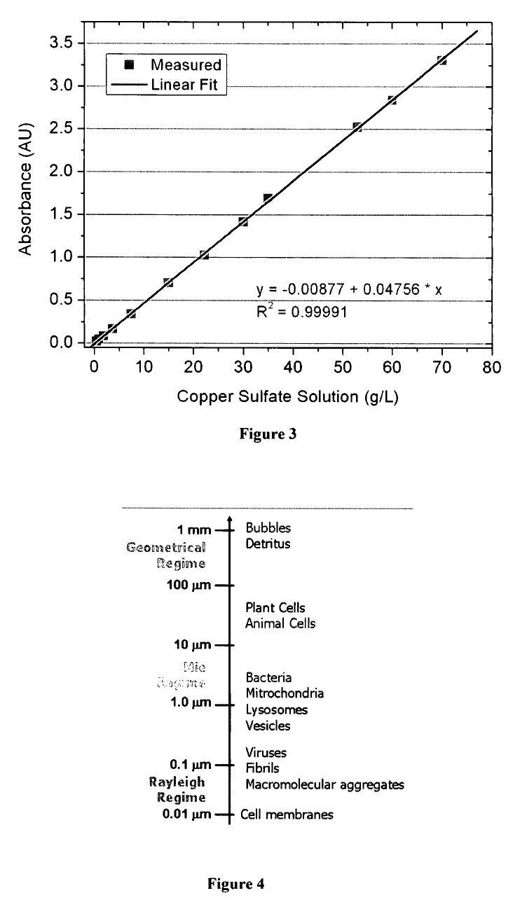

These working curves are obtained by measuring the signal from a series of standards of known concentration. The working curves can then be used to determine the concentration of an unknown sample or to calibrate the linearity of the analytical instrument. An example of a working curve for aqueous copper sulfate solutions of varying concentration is shown in FIG. 3.

Note that the Beer-Lambert law holds only if the sample (analyte) being measured has a well-defined absorption feature, and the spectral bandwidth of the light is relatively narrow compared to the line width of the sample's absorption feature. Furthermore, the Beer-Lambert Law assumes that the source radiation used by the measurement instrument is both monochromatic and collimated. Finally, there is also the assumption that the sample medium is homogeneous and free of multiple scattering events. Strictly speaking, for Beer-Lambert to hold—the light measured (e.g.: photons hitting the detector) must be only the light that has not been scattered. In many cases, especially those involving biological samples, scattering losses can sometimes dominate over absorbance, leading to a non-linear relationship between the sample concentration and the measured optical loss.

In general, common sources of non-linearity i.e., deviations in the relationship between optical loss and concentration (i.e., where Beer's Law cannot be directly applied) include:
multiple scattering events due to a high density of particulates in the sample,
changes in the refractive index of the sample medium and effective OPL at high analyte concentrations,
fluorescence or phosphorescence of the sample,
deviations in absorption coefficients at high analyte concentrations (owing to electrostatic interactions between molecules in close proximity),
shifts in chemical equilibrium as a function of concentration,
stray light entering the measurement system, and
non-monochromatic radiation
Note that deviations can be minimized by using a relatively flat part of the absorption spectrum such as the maximum of an absorption band.

### Scattering

For biological samples, especially those resulting from fermentation or cell culture, there is a very high degree of non-linearity owing to scattering. Scattering is a process by which small particles (such as cells, bacteria, or bubbles) suspended in a medium having a different index of refraction diffuse a portion of the incident radiation in all directions. Scattering changes the direction of light transport without changing its wavelength by “dispersing” the photons as they penetrate a turbid sample. The change in spatial distribution of the radiation is converted into a change in detected intensity by a photodiode which normally has both a fixed active area and location.

Scattering loss is a non-linear function of particle concentration, Δ, sample path length, OPL, particle diameter, d, wavelength, λ, and detector collection (cone) angle, θ, namely:

S=f(Δ, OPL, d, λ, θ). Eq. (6)

The scattering loss varies as a function of the ratio of the particle diameter, d, to the wavelength of the radiation, λ. FIG. 4 shows the relative sizes of most biological particles.

When this ratio is:
(d/λ)<0.1, the mechanism is Rayleigh scattering, in which the scattering coefficient varies inversely as the fourth power of the wavelength,
0.1<(d/λ)<10, the scattering varies in a complex fashion described by the Mie theory,
(d/λ)>10, the more simple laws of geometric optics can be applied.

Because of the many combinations of particle size, shape, and color, similar scattering coefficient readings can be obtained from samples containing physically distinct particles. Furthermore, the scattering coefficient is significantly influenced by the size distribution of the particles. In most real-world applications, there will be multiple particles having different sizes, indices of refraction, and concentrations. The overall scattering coefficient will be a composite function of all these parameters.

From FIG. 4, one can see that most suspended bioparticles (e.g.,cells, bacteria) and bubbles in aqueous bioreactor media are somewhat larger than the preferred optical density operating wavelength of λ=830 nm, so that the application of Mie theory is appropriate. Typically, bioparticles scatter about half the incident light energy into a 10-degree forward-directed cone (the active collection area of most optical density detectors), and less than 2.5 percent of it in the backward direction. The optical density sensor can collect scattered light within a 30-degree forward-directed cone, which maximizes detection of the transmitted light intensity. This large collection angle allows the sensor to achieve a high signal to noise ratio even in highly turbid media.

At a fixed wavelength and collection angle, the relationship between scattering loss and particle concentration (or scattering loss and sample path length) generally becomes non-linear when the scattering loss significantly exceeds 1.0 AU. In general, this deviation from linearity occurs when multiple scattering events become prevalent. This relationship depends on the size and optical properties of the particles.

At a fixed collection angle, for scattering losses below ˜1.0 AU, the scattering loss can usually be linearly related to a scattering coefficient, μ_{σ}, and OPL by:

S(λ)=μ_{σ}(λ, Δ, d)*OPL Eq. (7)

The scattering coefficient, μ_{σ} (λ, Δ, d) is equal to the fraction of energy dispersed from a light beam per unit of distance traveled in a scattering medium, in cm⁻¹. For example, a liquid having μ_{σ}=1 cm⁻¹ will scatter 90% of the energy out of a light beam over a distance of 1 cm, which corresponds to S=1 AU. Note that here we have used the same units for both scattering and absorbance. Although this notation is not strictly rigorous, as will be illustrated further below it can nonetheless be justified mathematically in the operating regime of the optical density sensor. The scattering coefficient of pure water at λ=830 nm is less than 0.0013 cm⁻¹ (primarily due to density fluctuations) and this can be negated by zeroing in pure water during primary calibration of the sensor. At a fixed wavelength and collection angle, for low particle concentrations, the scattering coefficient is proportional to particle concentration, C, namely, μ_{σ} (λ, C, d)=ξ_{scatter}(λ)*C. The scattering coefficient can also be related to the number density of particles, N, through the scattering cross section, σ_{scatter}, namely:

μ(λ, N)=σ_{scatter}(λ)*N. Eq. 8

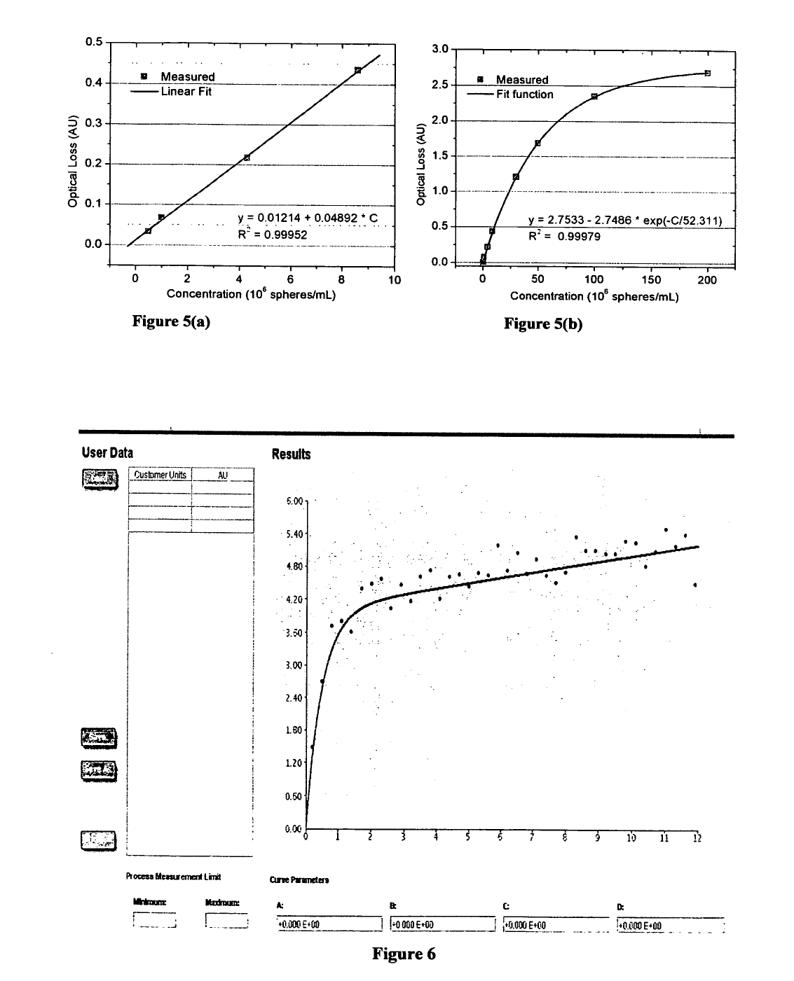

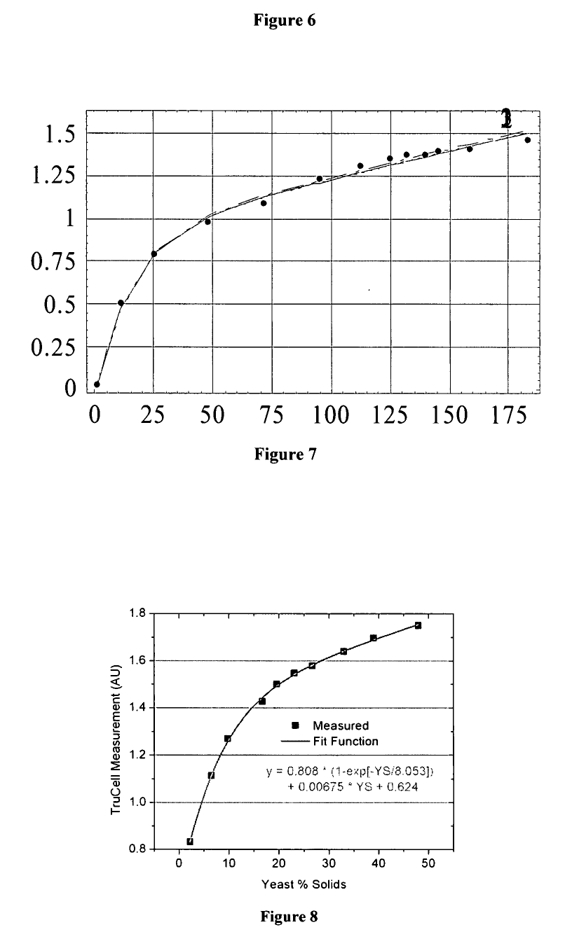

FIGS. 5a and 5b illustrates the dependence of scattering loss on concentration for 3 micron diameter polystyrene spheres in water. The measurements were made using a fixed path length of 10 mm, a 30 degree forward-scattering collection angle, and an excitation wavelength of 830 nm. In FIG. 5a, the scattering loss was measured for total optical losses of less than 0.5 AU, and the relationship between optical loss and concentration is indeed seen to be linear. In FIG. 5b, the scattering loss was measured for total optical losses significantly exceeding 1.0 AU, and the relationship is highly non-linear.

This nonlinearity, or deviation from the Beer-Lambert Law, is due to the forward scattering of the light combined with the collection geometry of the photo-detector. As described by Mie scattering, the incident light is scattered in all directions around the particle. The weighting of how much light is scattered at each particular angle can be calculated from fundamental electro-magnetic theory. In the Mie regime, the light is heavily forward scattered. For Beer-Lambert to strictly hold, each photon that scatters off a particle would need to be completely scattered out of the field of view of the detector. This is not the physical reality of the situation.

In lightly scattering media the amount of light forward or multiply scattered into the detector is very small compared to the light that reaches the detector without being scattered. Therefore, the relationship measured between particle density and scattering loss appears to be linear in lightly scattering media (typically, Lₒₚₜ<1.0 AU), as is illustrated in FIG. 5a. However, as the scattering density increases, the light that is scattered out of the field of view of the detector increases, while simultaneously the absolute amount of forward scattered light increases. As the scattering density increases, the amount of forward scattered light eventually reaches the same order of magnitude as the light directly reaching the detector. In this instance, the scattering loss appears to saturate, as illustrated in FIG. 5b. This saturation is observed in optical density measurements where Lₒₚₜ>˜1.0 AU, independent of the cell type (e.g., yeast, E. Coli, bacillovirus, etc).

For an optical density sensor the scattering loss saturates exponentially as a function of particle concentration:

AU(Δ)=A₁*[1−exp{−Δ/Δ₀}]+A₀ Eq. (9)

The physical basis for this deviation from linearity (versus absorbance) was described above. A more mathematical explanation of this phenomenon can be obtained through use of the Radiative Transfer Equation. This heuristic equation was introduced by Chandrakesar in 1950 [Radiative Transfer (1950, Clarendon Press, Oxford; reprinted by Dover Publications, Inc., 1960)] and was initially utilized to describe the transfer of radiation through interstellar space. It has also found use in describing the transfer of radiation in atmospheric and oceanic environments. A simplified version of the Radiative Transfer Equation is expressed below:

$$( \frac{1}{c} \frac{\partial }{\partial t} + \hat{n} \cdot ∇ ) L ( r ) = - ξ L ( r ) + \frac{s}{4 π} \int L ( r ) β ( θ , φ ) d Ω \qquad Eq . ( 10 )$$

In Equation 10, L(r) is radiance at a single wavelength (monochromatic radiation) at position r, n is a unit vector in the direction of the scattered ray, c is the speed of light in the medium, dΩ is the solid angle integration differential θ and φ are the spherical coordinate system radial and azimuthal angles, β(θ, φ) dΩ represents the probability that an incoming photon is scattered into the solid angle dΩ, ξ is the sum of the absorption (a) and scattering (s) coefficients in units of inverse length.

The left side of Equation 10 describes the propagation of the light, with the first term in parenthesis giving the time dependence and the second term giving the spatial dependence. The first term on the right of the equals sign describes the scattering and absorptive losses. The second term on the right describes the fraction of the total scattered light that can be collected by the detector.

The detector has a limited aperture and acceptance angle, and these factors limit the amount of the propagating and scattered light that will actually be recorded at the observation point by the detector. Under steady state conditions

$$( \frac{1}{c} \frac{\partial }{\partial t} L ( r ) = 0 )$$

and when scattering is negligible (s˜0), the radiative transfer equation reduces in one dimension to:

L(x)/L(0)=e^{-ξx} Eq. (11)

This is the Beer-Lambert Law, but expressed using radiance, L(x), instead of intensity, 1. Specifically, under steady state conditions, if the detector's field of view, as defined by the angles θ and φ, is very small then the integral is close to zero and the radiative transfer equation again reduces to the Beer-Lambert law, and a linear relationship between concentration and optical loss can be extended. In general, the integral is finite and a saturation of the Beer-Lambert Law is observed. This is because as the scatterer density becomes high, the power in the forward scattered light approaches the same order of magnitude as the transmitted light. The more the detector is apertured, the less forward scattered light enters the detector and the higher the concentration of scatterers for which the Beer Lambert law holds.

When absorbance measurements are being made in real-time inside a bioreactor, the measurement is subject to the effects of agitation and sparging, in that the liquid medium can contain bubbles, floating detritus and suspended cells or bacteria. These dynamic effects present during in-reactor measurements may further add to the scattering and absorbance losses of the cells themselves, and confound the optical loss measurement. Care should be taken to ensure that the optical density sensor is completely covered by the cell medium, but is placed at a distance from the sparger to avoid affecting the measurement by bubbles from the sparger. In addition to absorption and scattering losses, an optical transmission sensor can encounter other types of optical losses which are typically non-linear, and may distort the measurement. We note that a laser-based sensor having optical windows whose index of refraction is matched to that of the bioreactor medium will avoid most of these potential pitfalls. However, a lamp or LED-based sensor having sapphire windows will generally not perform as well. A typical lamp or LED light source used to measure absorbance can have an optical bandwidth of 20 to 50 nm, and therefore will produce a measurement that is a convolution of the optical response over a wide variety of wavelengths. Such a response can often lead to non-linear sensor performance. If the lamp or LED-based cell density sensor has any variation in its operating wavelength, an even greater spread in the optical measurements may be obtained. For biological samples, especially those related to fermentation or cell culture, there is a very high degree of non-linearity owing to scattering processes. For optical density sensor measurement, we have determined that the primary optical loss mechanism will be scattering, and that in most growth runs, a high optical loss will be reached. Therefore, we expect an exponential saturation of the optical loss measurements owing to significant forward scattering saturating the photodetector response.

### DESCRIPTION OF THE INVENTION

As already indicated, cells can sometimes produce as much scattering as absorption, so the raw optical loss data (normally expressed in AU) will sometimes not follow the Beer Lambert Law in cellular media. In such cases, the relationship between raw AU and cell density will therefore not be linear. Also, because an optical density detector has a non-negligible field of view, the optical density sensor will also tend to not follow the Beer-Lambert Law as the scattering density increases. We have developed a fitting equation (Equation 12) that overcomes to a significant extent the problem of the deviation from the Beer-Lambert Law. Additionally, our fitting equation addresses the collection of forward scattered light by the sensor and the apparent saturation of the optical loss that results from such collection since the terms of our equation account for the saturation. We have found that a fitting function of measured optical loss versus a process parameter such as cell density will have the mathematical form:

$${y}_{ol} = A + B ( 1 - {e}^{- \frac{{x}_{PU}}{C}} ) + D \cdot {x}_{PU} \qquad Eq . ( 12 )$$

wherein x_{PU} is expressed in the process units (PU) such as weight(mg/l) or number density (#/volume), yₒₗ is optical loss in the chosen units (normally AU). A, B, C and D are fitting coefficients wherein A is the offset constant which is determined by curve fitting to the data, B is the effective scattering amplitude, C is the scattering coefficient and D is the absorption coefficient. Our fitting function is particularly advantageous for applications such as bacterial growth where the cell concentration can become high, so that that the scattering loss will tend to dominate.

A conversion program (a curve fitting algorithm) that uses the mathematical fitting function described in Equation (12) to generate the parameters for the curve fit permits the optical density transmitter to directly convert raw AU data into user-defined process units. By doing this in real-time, end users can generate meaningful in situ process data for controlling their bioreactor process. For example, the conversion program can be used to convert from raw AU to cell density (cells/mL), optical density (OD), or dry cell weight (mg/L).

The conversion program receives a file containing both AU (when yₒₗ is measured in AU) and process data (x_{PU}) from a growth run, and fits that data to Equation (12). A Levenberg-Marquardt algorithm is most preferably used to perform the curve fit. Other suitable fitting algorithms include “learning algorithm” or other non-linear least squares method algorithms. In general, the accuracy and reliability of the curve fit algorithm is enhanced if the user also supplies the program with minimum and maximum expected process values (range of obtained data values) for their process measurements. The plot range of the fitted function produced can be set to these values (which can sometimes extend beyond the measured data range).

Process units are often very large units (such as millions of cells/mL). Such large numbers can sometimes generate problems for fitting algorithms, so in use our chosen fitting algorithm will preferably first scale the user data before fitting. This scaling serves to maintain the maximum effective x_{PU}-value below 100. The scale factor is derived by taking the base 10 logarithm of the maximum that the process value that is recorded. This base 10 logarithm of the maximum process value can be expressed as log₁₀(processMax). If this number is smaller than 2, a scale factor of unity is applied. If this number is greater than 2, the x_{PU}-values are divided by:

10^{(floor(log}^{₁₀}^{(processMax)-2))}

The curve fit coefficients can be scaled in a like manner.

FIG. (6) shows an embodiment of such a program. This Figure shows how a user uses the curve fit algorithm: (in this case a Levenberg-Marquardt algorithm)
1. Enter the data into the data field by cutting and pasting or by reading in a comma delimited file (CSV format) using the “Load” button.
2. Specify the minimum and maximum expected process values.
   a. The data and the minimum/maximum process values can be stored by pressing the left “Save” or “Save As” button.
3. Press the “Run” button to perform the fit and plot the data and the fitting curve.
4. The fitting parameters are then shown on the screen.
   a. The “Save” button on the right will record and save the data, fitting parameters, and the user process range minimum and maximum.

### Accuracy of Curve Fit

Both consistency and performance of the curve fitt algorithm for measuring optical density in accordance with the present invention are important. Fits based on a set of twelve (12) diverse bioprocesses have established and confirmed the applicability of the fitting function (Equation 12) in accordance with the present invention. Essentially all of these fits had R² values, or fit figures of merit, greater than 0.99×, and generally greater than 0.999×. Accuracy testing demonstrated that 4 significant digits in the fitting parameters (A, B, C, D), as shown in Equation (12), are sufficient to reduce errors in the measurement of both optical loss and other process units to below 1%. The accuracy tests also showed that the “B” parameter is the most sensitive to the number of significant digits, followed by the “D” and “C” parameters. A 0.1% error in all fitting parameters produced a worst case 0.1% error in the final AU values. Similarly, a 1% error in all fitting parameters produced a worst case 1% error in the final AU values. Note that a 1% error in a fitting parameter is a change only in the 3^{rd} significant digit. Therefore, changes in the 4^{th} significant digit will limit the fitting errors to below 1%, namely the precision specification of the optical density sensor. An example of one case used to analyze the effects of errors in the fitting parameters on the conversion curve is presented in FIG. 7 which shows the original fit, the fit with a worst case error in all parameters of 0.1%, and the fit with a worst case error in all parameters of 1%. Only the 1% error curve is even visible.

Various test runs were also tabulated to show the change in AU value. As can be seen from the table below, the AU errors are insignificant until the parameter errors reach 1%. The significant effect on the results of the “B” parameter occurs because most of these data sets exhibit a saturation curve. The term multiplying the “B” parameter gives the shape of the curve and the “B” parameter sets the scaling.

| Fit | Rounded 4sig | Rounded 3sig | 0.1% error | 1% error |
|---|---|---|---|---|
| 0.0319727 | 0.0319766 | 0.0319903 | 0.0319465 | 0.0316902 |
| 0.473951 | 0.473976 | 0.47403 | 0.474037 | 0.474635 |
| 0.800706 | 0.800726 | 0.800839 | 0.801135 | 0.804908 |
| 1.01246 | 1.01246 | 1.01267 | 1.01328 | 1.02079 |
| 1.12483 | 1.12483 | 1.12513 | 1.12586 | 1.13542 |
| 1.20941 | 1.20941 | 1.2098 | 1.21055 | 1.22126 |
| 1.26943 | 1.26943 | 1.2699 | 1.27065 | 1.282 |
| 1.3104 | 1.3104 | 1.31091 | 1.31165 | 1.32342 |
| 1.33514 | 1.33514 | 1.33568 | 1.33642 | 1.34843 |
| 1.36183 | 1.36183 | 1.36241 | 1.36313 | 1.3754 |
| 1.37782 | 1.37783 | 1.37842 | 1.37915 | 1.39157 |
| 1.42377 | 1.42378 | 1.42443 | 1.42514 | 1.438 |
| 1.50429 | 1.5043 | 1.50505 | 1.50573 | 1.51934 |

### Examples of Curve Fitting for Different Processes

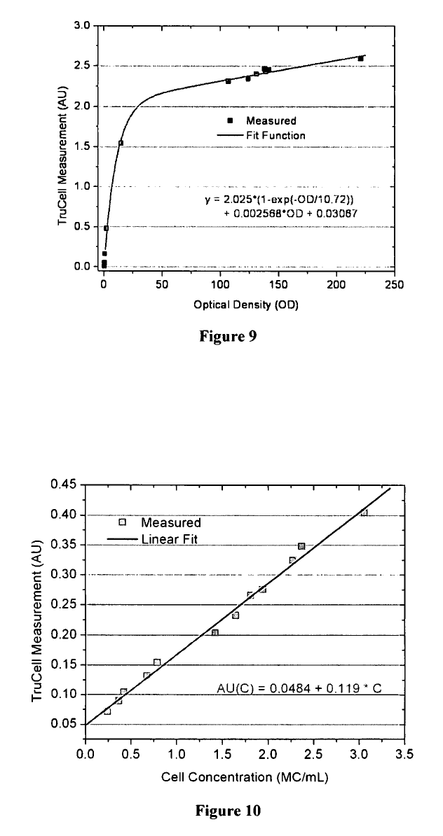

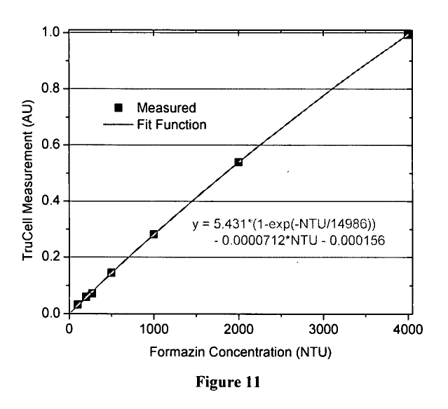

FIGS. 8 through 11 illustrate examples of the fitting function as applied to fermentation (yeast and E. Coli), mammalian cell culture, and insect culture. The AU values have been fitted against cell density, dry cell weight, and optical density process parameters.

FIG. (8) shows the response of an optical density sensor to high concentrations of yeast slurry as used in beer pitching. The optical loss becomes high at high yeast concentrations, and comprises both absorption and scattering loss mechanisms. The measurement also has a significant initial offset. Note the close fit of the measured data to the general mathematical form for optical loss: Equation 12 works well to convert optical loss (AU) to process units of “yeast % solids”.

FIG. (9) shows the response of an optical density sensor to typical concentrations of E. Coli during fermentation. The concentration of E. Coli is measured using optical density (OD) units. The optical loss becomes very high (even higher than for the yeast in FIG. 8) at the end of the growth run, and the optical response begins to saturate. The measurement only has a small initial offset. Note the excellent correlation provided by Equation 12 between AU and OD.

FIG. (10) shows the response of a Optical density sensor to typical concentrations of Chinese Hamster Ovary (CHO) mammalian cells during a cell culture run. The optical loss remains low, and the fit function can be approximated by a line. The measurement only has a small initial offset. Note also the close fit of the measured data to the linearized mathematical form for optical loss.

FIG. (11) shows the response of an optical density sensor to formazin, whose concentration is typically measured in nephelometric turbidity units. The optical loss remains relatively low, and the fit function can be almost approximated by a line, although there is slightly more visible curvature than for the cell culture. Note that formazin has a 40% variation in its size distribution, and forms clusters, so that it provides another distinct example of a scattering medium for which equation 10 holds.

1. A method for converting non-linear optical loss readings in a bioreactor process vessel into process parameter units by applying a curve fitting algorithm to the fitting function represented by the empirical equation:
$$yol = A + B ( 1 - {e}^{- \frac{{x}_{PU}}{C}} ) + D \cdot {x}_{PU}$$
wherein x_{PU} is in the process units (PU), yₒₗ is optical loss in the chosen units, A is the offset constant, B is the effective scattering amplitude, C is the scattering coefficient constant and D is the absorption coefficient.

2. A method in accordance with claim 1 wherein the fitting algorithm is Levenberg-Marquardt

3. A method in accordance with claim 1 wherein the process parameters being measured are cell density, dry cell weight, and/or optical density

4. A method in accordance with claim 1 wherein the fitting coefficients have 4 significant digits
